(19) United States
(12) Patent Application Publication
(10) Pub. No.: US 2015/0335894 A1
(43) Pub. Date: Nov. 26, 2015

(54) COMMUNICATIONS SCHEME FOR DISTRIBUTED LEADLESS IMPLANTABLE MEDICAL DEVICES ENABLING MODE SWITCHING

(71) Applicant: PACESETTER, INC., Sylmar, CA (US)

(72) Inventors: Gene A. Bornzin, Simi Valley, CA (US); Didier Theret, Porter Ranch, CA (US)

(73) Assignee: PACESETTER, INC., Sylmar, CA (US)

(21) Appl. No.: 14/283,094

(22) Filed: May 20, 2014

(51) Int. Cl. A61N 1/368; A61N 1/375

(52) U.S. Cl. CPC A61N 1/3682; A61N 1/3756

(57) ABSTRACT

A distributed leadless implantable system is provided that comprises first and second leadless implantable medical devices (LIMD) configured to be implanted entirely within first and second chambers of the heart. Each of the first and second LIMDs comprises a housing having a proximal end configured to engage local tissue of interest in a local chamber, electrodes located along the housing and cardiac sensing circuitry configured to detect intrinsic and paced cardiac events occurring in a near field associated with the local chamber.

### FIELD OF THE INVENTION

Embodiments herein generally relate to implantable medical devices, and more particularly to leadless intra-cardiac implantable medical devices that afford dual chamber functionality. As used herein, the term “leadless” generally refers to an absence of electrically-conductive leads that traverse vessels or other anatomy outside of the intra-cardiac space, while “intra-cardiac” means generally, entirely within the heart and associated vessels, such as the SVC, IVC, CS, pulmonary arteries and the like.

### BACKGROUND OF THE INVENTION

Current implantable medical devices (IMD) for cardiac applications, such as pacemakers, include a “housing” or “can” and one or more electrically-conductive leads that connect to the can through an electro-mechanical connection. The can is implanted outside of the heart, in the pectoral region of a patient and contains electronics (e.g., a power source, microprocessor, capacitors, etc.) that provide pacemaker functionality. The leads traverse blood vessels between the can and heart chambers in order to position one or more electrodes carried by the leads within the heart, thereby allowing the device electronics to electrically excite or pace cardiac tissue and measure or sense myocardial electrical activity.

To sense atrial cardiac signals and to provide right atrial chamber stimulation therapy, the can is coupled to an implantable right atrial lead including at least one atrial tip electrode that typically is implanted in the patient's right atrial appendage. The right atrial lead may also include an atrial ring electrode to allow bipolar stimulation or sensing in combination with the atrial tip electrode.

Before implantation of the can into a subcutaneous pocket of the patient, however, an external pacing and measuring device known as a pacing system analyzer (PSA) is used to ensure adequate lead placement, maintain basic cardiac functions, and evaluate pacing parameters for an initial programming of the device. In other words, a PSA is a system analyzer that is used to test an implantable device, such as an implantable pacemaker.

To sense the left atrial and left ventricular cardiac signals and to provide left-chamber stimulation therapy, the can is coupled to the “coronary sinus” lead designed for placement in the “coronary sinus region” via the coronary sinus ostium in order to place a distal electrode adjacent to the left ventricle and additional electrode(s) adjacent to the left atrium. Accordingly, the coronary sinus lead is designed to: receive atrial and/or ventricular cardiac signals; deliver left ventricular pacing therapy using at least one left ventricular tip electrode for unipolar configurations or in combination with left ventricular ring electrode for bipolar configurations; deliver left atrial pacing therapy using at least one left atrial ring electrode as well as shocking therapy using at least one left atrial coil electrode.

To sense right atrial and right ventricular cardiac signals and to provide right-chamber stimulation therapy, the can is coupled to an implantable right ventricular lead including a right ventricular (RV) tip electrode, a right ventricular ring electrode, a right ventricular coil electrode, a superior vena cava (SVC) coil electrode, and so on. Typically, the right ventricular lead is inserted transvenously into the heart so as to place the right ventricular tip electrode in the right ventricular apex such that the RV coil electrode is positioned in the right ventricle and the SVC coil electrode will be positioned in the right atrium and/or superior vena cava. Accordingly, the right ventricular lead is capable of receiving cardiac signals, and delivering stimulation in the form of pacing and shock therapy to the right ventricle.

Although a portion of the leads are located within the heart, a substantial portion of the leads, as well as the can itself are outside of the patient's heart. Consequently, bacteria and the like may be introduced into the patient's heart through the leads, as well as the can, thereby increasing the risk of infection within the heart. Additionally, because the can is outside of the heart, the patient may be susceptible to Twiddler's syndrome, which is a condition caused by the shape and weight of the can itself. Twiddler's syndrome is typically-characterized by a subconscious, inadvertent, or deliberate rotation of the can within the subcutaneous pocket formed in the patient. In one example, a lead may retract and begin to wrap around the can. Also, leads may dislodge from the endocardium and cause the device to malfunction.

In addition to the foregoing complications, implanted leads may experience certain further complications, such as incidences of venous stenosis or thrombosis, device-related endocarditis, lead perforation of the tricuspid valve and concomitant tricuspid stenosis; and lacerations of the right atrium, superior vena cava, and innominate vein or pulmonary embolization of electrode fragments during lead extraction.

To combat the foregoing limitations and complications, small sized devices configured for intra-cardiac implant have been proposed. These devices, termed leadless pacemakers (LLPM), are typically characterized by the following features: they are devoid of leads that pass out of the heart to another component, such as a pacemaker can outside of the heart; they include electrodes that are affixed directly to the can of the device; the entire device is attached to the heart; and the device is capable of pacing and sensing in the chamber of the heart where it is implanted.

LLPM devices that have been proposed thus far offer limited functional capability. These LLPM devices are able to sense in one chamber and deliver pacing pulses in that same chamber, and thus offer single chamber functionality. For example, an LLPM device that is located in the right atrium would be limited to offering AAI mode functionality. An AAI mode LLPM can only sense in the right atrium, pace in the right atrium and inhibit pacing function when an intrinsic event is detected in the right atrium within a preset time limit.

Similarly, an LLPM device that is located in the right ventricle would be limited to offering VVI mode functionality. A VVI mode LLPM can only sense in the right ventricle, pace in the right ventricle and inhibit pacing function when an intrinsic event is detected in the right ventricle within a preset time limit. To gain widespread acceptance by clinicians, it would be highly desired for LLPM devices to have dual chamber pacing/sensing capability (DDD mode) along with other features, such as rate adaptive pacing.

It has been proposed to implant sets of multiple LLPM devices within a single patient, such as one or more LLPM devices located in the right atrium and one or more LLPM devices located in the right ventricle. The atrial LLPM devices and the ventricular LLPM devices wirelessly communicate with one another to convey pacing and sensing information there between to coordinate pacing and sensing operations between the various LLPM devices.

However, these sets of multiple LLPM devices experience various limitations. For example, each of the LLPM devices must expend significant power to maintain the wireless communications links. The wireless communications links should be maintained continuously in order to constantly convey pacing and sensing information between, for example, atrial LLPM device(s) and ventricular LLPM device(s). This pacing and sensing information is necessary to maintain continuous synchronous operation, which in turn draws a large amount of battery power.

Further, it is difficult to maintain a reliable wireless communications link between LLPM devices. The LLPM devices utilize low power transceivers that are located in a constantly changing environment within the associated heart chamber. The transmission characteristics of the environment surrounding the LLPM device change due in part to the continuous cyclical motion of the heart and change in blood volume. Hence, the potential exists that the communications link is broken or intermittent.

### SUMMARY

In accordance with an embodiment, a distributed leadless implantable system is provided that comprises first and second leadless implantable medical devices (LIMD) configured to be implanted entirely within first and second chambers of the heart. Each of the first and second LIMDs comprises a housing having a proximal end configured to engage local tissue of interest in a local chamber, electrodes located along the housing and cardiac sensing circuitry configured to detect intrinsic and paced cardiac events occurring in a near field associated with the local chamber. Each of the first and second LIMDs also comprises a controller configured to analyze the intrinsic and paced events and, based thereon, produce a trigger pulse at the electrodes when an event of interest occurs in the local chamber. Each of the first and second LIMDs also comprises pulse sensing circuitry configured to detect at least one of a paced event or a trigger pulse occurring in a far field, where the paced event or trigger pulse originates in a remote chamber that differs from the corresponding local chamber, the trigger pulse having a predetermined pattern configured to indicate that an event of interest has occurred in the remote chamber. The controller recognizes at least one of the paced event or the trigger pulse to indicate an occurrence of the event of interest in the remote chamber, and in response thereto, initiates a related action in the local chamber.

Optionally, the system may locate the first LIMD in an atrium and the second LIMD in a ventricle, the first LIMD producing an AS/AP trigger, as the trigger pulse, to indicate that an atrial sensed event or atrial paced event occurred. Alternatively, the system may locate the first LIMD in an atrium and the second LIMD in a ventricle, with the second LIMD producing a VS/VP trigger, as the trigger pulse, to indicate that a ventricular sensed event or ventricular paced event occurred. Optionally, the first LIMD produces an AV end trigger, as the trigger pulse, and the second LIMD detects and recognizes the AV end trigger to indicate an end of an AV delay. The second LIMD may deliver a pacing pulse upon detecting the AV end trigger, unless the second LIMD detected a corresponding intrinsic ventricular event before detecting the AV end trigger.

Alternatively, the first LIMD produces an AV start trigger, as the trigger pulse, and the second LIMD detects and recognizes the AV start trigger as an instruction for the second LIMD to start an AV delay. Optionally, the second LIMD produces a VA end trigger, as the trigger pulse, and the first LIMD detects and recognizes the VA end trigger to indicate an end of a VA delay. In this last instance, the first LIMD is configured to deliver a pacing pulse upon detecting the VA end trigger, unless the first LIMD detects a corresponding intrinsic atrial event before detecting the VA end trigger.

Optionally, the second LIMD produces a post ventricular-atrial refractory period start trigger, as the trigger pulse, and the first LIMD detects and recognizes the PVARP start trigger as an instruction for the first LIMD to start a PVARP delay. Alternatively, the system may include the first LIMD being located in an atrium and the second LIMD being located in a ventricle, with the first LIMD set in an AAI mode with a first base rate and the second LIMD set in a VVI model with a second base rate that is lower than the first base rate. In this last example, the second LIMD ignores atrial pulses from the first LIMD.

Alternatively, the controller may produce the trigger pulses formatted in a manner that distinguishes the trigger pulses from pacing pulses based on at least one of amplitude, pulse width, inter-pulse delay, pulse frequency, or pulse morphology. Optionally, when the first LIMD in the atrium is turned off or fails, the second LIMD in the ventricle may be switched to a function in a VVI pacing mode.

In accordance with another embodiment, a method is described for providing an implantable cardiac system that comprises providing first and second leadless implantable medical devices (LIMD) configured to be implanted entirely within first and second chambers of the heart, where the first and second LIMD have electrodes on housings thereof to engage local tissue of interest in a corresponding first and second chambers. The method detects, at the first LIMD, at least one of intrinsic or paced events originating in the first chamber. The method analyzes, at the first LIMD, the intrinsic and paced events and, based thereon, produces a trigger pulse at the electrodes of the first LIMD when an event of interest occurs in the first chamber. The method detects, at the second LIMD in the second chamber, at least one of a paced event or the trigger pulse that was produced by the first LIMD in a far field (FF) within the first chamber, where the at least one of the paced event or trigger pulse originates in a remote chamber that differs from the corresponding local chamber. The trigger pulse has a predetermined pattern configured to indicate that an event of interest has occurred in the remote chamber. The method recognizes the at least one of the paced event or trigger pulse to indicate an occurrence of the event of interest in the remote chamber, and in response thereto, initiates a related action in the local chamber.

Optionally, the method may comprise locating the first LIMD in an atrium and locating the second LIMD in a ventricle, and producing an AS/AP trigger from the first LIMD, as the trigger pulse, to indicate that an atrial sensed (AS) event or atrial paced (AP) event occurred. Optionally, the method may further comprise locating the first LIMD in an atrium and locating the second LIMD in a ventricle, and producing a VSNP trigger from the second LIMD, to indicate that a ventricular sensed (VS) event or ventricular paced (VP) event occurred. Optionally, the method may comprise producing an AV end trigger, detecting and recognizing the AV end trigger to indicate an end of an AV delay, and delivering a pacing pulse upon detecting the AV end trigger, unless a corresponding intrinsic ventricular event is detected before detecting the AV end trigger.

Optionally, the method may comprise producing an AV start trigger, as the trigger pulse, and detecting and recognizing the AV start trigger as an instruction for the second LIMD to start an AV delay. The method may comprise producing a VA end trigger, as the trigger pulse, detecting and recognizing the VA end trigger to indicate an end of a VA delay, and delivering a pacing pulse upon detecting the VA end trigger, unless the first LIMD detected a corresponding intrinsic atrial event before detecting the VA end trigger. Optionally, the method may comprise producing a post ventricular-atrial refractory period (PVARP) start trigger, as the trigger pulse, and detecting and recognizing the PVARP start trigger as an instruction for the first LIMD to start a PVARP delay.

Alternatively, the method may comprise setting the first LIMD in an AAI mode with a first base rate and setting the second LIMD in a WI mode with a second base rate that is lower than the first base rate, the second LIMD ignoring atrial pulses from the first LIMD. Optionally, the method may comprise, when the first LIMD is turned off or fails, changing a mode of operation of the second LIMD from a DDD pacing mode to a WI pacing mode.

### BRIEF DESCRIPTION OF THE DRAWINGS

FIG. 1 illustrates a graphical representation of a heart with an implantable medical system located therein in connection with providing pacing therapy, cardiac rhythm management (CRP) therapy and the like.

FIG. 2 illustrates a simple block diagram of at least a portion of the circuitry within an LIMD.

FIG. 3 illustrates a method in accordance with an embodiment herein for managing communication between LIMDs in connection with providing select modes of operation, such as a DDD mode.

FIG. 4 illustrates a timing diagram followed in accordance with a distributed communications embodiment.

FIG. 5 illustrates timing diagrams that may be implemented in accordance with an embodiment.

FIG. 6 illustrates timing diagrams associated with a condition in which the physiologic intrinsic rate is higher than the programmed rate but where spontaneous R-waves occur before the end of the AV delay.

FIG. 7 illustrates the timing diagrams corresponding to atrial and ventricular devices operating in a DDD mode, when a premature ventricular contraction occurs.

FIG. 8 illustrates timing diagrams corresponding to the operation of atrial and ventricular devices configured to operate in accordance with an alternative embodiment.

FIG. 9 illustrates a flow chart of the system behavior at different atrial rates in accordance with an embodiment.

FIG. 10 illustrates a side perspective view of an LIMD oriented with the base facing upward.

### DETAILED DESCRIPTION

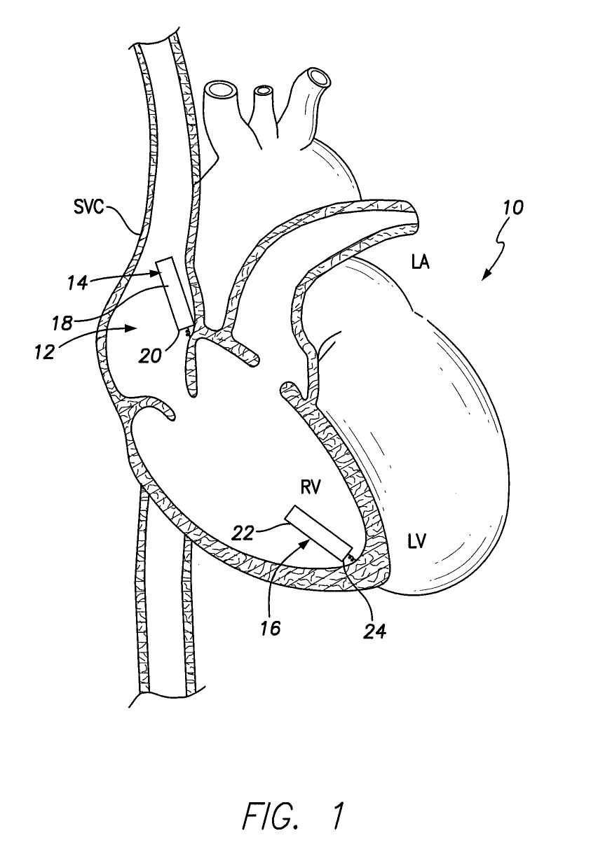

FIG. 1 illustrates a graphical representation of a heart 10 with an implantable medical system 12 located therein in connection with providing pacing therapy, cardiac rhythm management (CRT) therapy and the like. The system 12 includes at least first and second leadless implantable medical devices (LIMD) 14 and 16 that are configured to be implanted entirely within corresponding first and second chambers of the heart. In the example of FIG. 1, the LIMD 14 is implanted in the right atrium, while the LIMD 16 is implanted in the right ventricle. Optionally, additional LIMDs may be implanted in the left atrium and/or the left ventricle. Alternatively, the LIMDs 14 and 16 may be implanted in other combinations of chambers, such as the LIMD 14 in the right atrium, while the LIMD 16 is in the left atrium. Optionally, the LIMDs 14 and 16 may be located in the left atrium and left ventricle, respectively, or in the right ventricle and left ventricle, respectively, and the like.

Each of the LIMDs 14 and 16 (also referred to as atrial or ventricular pacing devices based on implant location) are constructed with a generally similar hardware configuration having a housing 18, 22 with a proximal end 20, 24 that is configured to engage local tissue of interest within the corresponding local chamber. For example, the LIMD 14 has a proximal end 20 that is configured to engage local tissue in the right atrium, while the LIMD 16 has a proximal end 24 configured to engage local tissue in the right ventricle. Electrodes (not illustrated in FIG. 1) may be located along the housings 18 and 22 in various positions and combinations. The internal electrical components and electrodes may be implemented as described in U.S. patent application Ser. No. 13/653,248, filed Oct. 16, 2012 (U.S. Published Application No. 20140107723), the complete subject matter of which are expressly incorporated herein by reference in its entirety.

As explained hereafter, the system 12 provides a simple communications scheme that may be used to provide a distributed leadless pacing system that may switch between DDD mode and other modes, as explained hereafter. The distributed system 12 has multiple LIMDs 14 and 16 that operate as separate pacing devices, such as separate atrial and ventricular pacing devices. The atrial and ventricular pacing devices include two types of sensing configurations, namely a sensing channel tuned to detect intrinsic and/or paced cardiac events. For example, the cardiac event sensing channel, for an atrial pacing device, may be tuned and function to sense P-waves or paced events delivered in the atrium. A ventricular pacing device may be tuned and function to sense R-waves or paced events delivered in the ventricle over the cardiac event sensing channel.

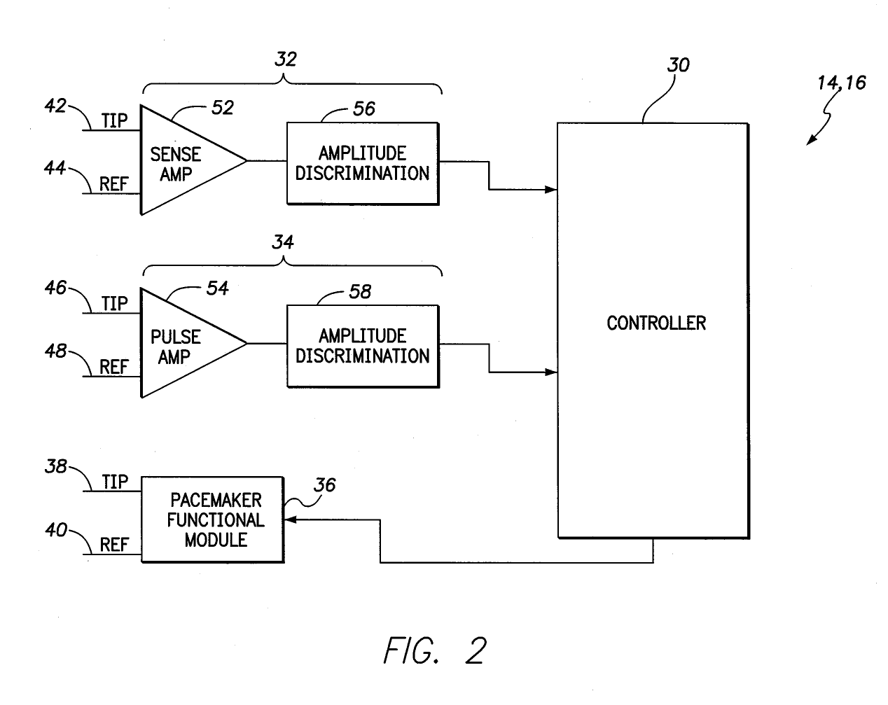

FIG. 2 illustrates a simple block diagram of at least a portion of the circuitry within an LIMD 14, 16. The LIMD 14, 16 includes a controller 30 that is coupled to cardiac sensing circuitry 32 and pulse sensing circuitry 34. The controller 30 also utilizes or communicates with various other electronic components, firmware, software and the like that generally perform sensing and pacing functions (as generally denoted by a pacemaker functional block 36). While the examples herein are provided for pacing functions, the same distributed LIMDs could be programmed to perform anti-tachycardia pacing, cardiac rhythm therapy and the like. The cardiac sensing circuitry 32 is configured to detect intrinsic and paced cardiac events that occur in a near field (NF) associated with a local chamber in which the LIMD 14, 16 is implanted. The pulse sensing circuitry 34 is configured to detect trigger pulses that occur in a far field (FF) associated with a remote chamber. The trigger pulses originate from an LIMD 14, 16 that is located in a remote different chamber of the heart and that is separate from the local chamber in which a particular LIMD is located. The trigger pulses have a predetermined pattern configured to indicate that an event of interest has occurred in the remote chamber. The event of interest may represent a physiologic event, either intrinsic or externally induced (e.g., paced). The event of interest may also represent a non-physiologic action, such as the initiation or termination of a timer as used in connection with various operations of a pacemaker, and the like.

The controller 30 is configured to analyze incoming NF intrinsic and/or paced cardiac events (as sensed over the cardiac sensing circuitry 32). Based on this analysis, the controller 30 performs various pacemaker related actions, such as setting or ending timers, recording data, delivery of therapy and the like. The controller 30 also produces one or more trigger pulses that are output from the electrodes of the LIMD 14, 16. In the example of FIG. 2, inputs 38 and 40 represent output terminals that are coupled through a switching circuit (in the functional module 36) to corresponding electrodes on the housing of the LIMD 14, 16.

Inputs 42-48 are provided to the cardiac and pulse sensing circuitry 32 and 34. By way of example, inputs 42 and 44 may be coupled to tip and reference electrodes that supply sensed signals to a sensing amplifier 52. Inputs 46 and 48 may be coupled to the same or different tip and reference electrodes to provide sensed signals to a pulse amplifier 54. An output of the sensing amplifier 52 is supplied to amplitude discriminator 56, while an output of the pulse amplifier 54 is supplied to amplitude discriminator 58. Outputs of the amplitude discriminators 56 and 58 are then provided to the controller 30 for subsequent analysis and appropriate actions.

By way of example, the sensing amp 52 and discriminator 56 may be tuned to perform NF atrial sensing and have programmable or automatic blanking periods. The blanking period is set to avoid sensing far field ventricular events at the sensing amp 52. When the LIMD 14, 16 represents a ventricular device, the sensing amp 52 and discriminator 56 may be tuned or configured to sense R-waves occurring in the local ventricle. For example, the amp 52 may be programmed to a select gain, while the discriminator 56 is programmed to only pass signals that exceed a select sensing threshold. Optionally, the discriminator 56 may include a band pass, low pass or high pass filter set to only pass signals within a select frequency range. The amp 52 and/or discriminator 56 may have a low pass frequency (e.g., 10-120 Hz). The gain, threshold and/or pass band may be adjusted for atrial versus ventricular devices.

The pulse amplifier 54 and amplitude discriminator 58 are configured to detect select communication pulses having one or more known predetermined formats. For example, the gain of the amp 54 and threshold of the discriminator 58 may be set to pass only signals below a select pulse maximum threshold. Optionally, the discriminator 58 may include a band pass, low pass or high pass filter set to only pass pulses within a select frequency range. The amp 54 and/or discriminator 58 may have a high pass frequency (e.g., 500 Hz-10 KHz). The pulse amp 54 and amplitude discriminator 58 may also be configured to sense pacing pulses delivered in the local and/or remote chambers. The communications pulses sensed by the pulse amp 54 and amplitude discriminator 58 represent trigger pulses that are delivered to the controller 30 and used to indicate different events of interest (e.g., physiologic and non-physiologic events or actions). The controller 30 then takes appropriate action, depending upon the situation.

Optionally, a single amplifier may be used in place of amps 52 and 54, thereby detecting low and high frequency signals. An output of the single amp may be coupled to a low pass filter in parallel with a high pass filter that separate the low and high frequency components, respectively.

It should be noted that the LIMDs 14 and 16 may be constructed with substantially identical external and internal architectures, but for portions of the firmware that are programmed differently between the LIMDs 14, 16 based on which chamber of the heart the device is located. For example, for an atrial device, the firmware may be set such that the atrial pulse amplifier has the purpose of detecting special ventricular trigger pulses signaling spontaneous ventricular events that occur during the ventricular alert period or events that occur during the AV delay. The trigger pulses may have various predetermined shapes, amplitudes, pulse widths, pulse patterns and the like. Different pulse formulations may be assigned different meanings to instruct receiving atrial or ventricular device to take difference actions. The pulse amplitude may be below a capture threshold that might otherwise achieve capture of heart tissue.

As an example, small, subthreshold (e.g., below the capture threshold) multipulse signals (e.g., double pulse, triple pulse and the like) may be used to trigger the atrial pacing device to respond to select ventricular events. As an example, when a trigger pulse is detected, an atrial device may respond by starting a PVARP interval timer. The pulse sensing circuitry 34 is configured to discriminate (block) conventional pulse configurations that are too large in amplitude and/or do not exhibit the desired pulse frequency or combination (e.g., a double pulse pair). During operation, following an atrial event, when an intrinsic ventricular event does not occur within the appropriate time, a V pace pulse is delivered at the end of the AV delay from the ventricular device. The V pace pulse is delivered from the ventricular device when the state machine in the atrial device determines that a ventricular pacing event should occur.

Optionally, it should be noted that far field intrinsic R-waves may be detected at an atrial device and used to detect ventricular events. When doing so, a process may be implemented in the firmware of the atrial and ventricular devices to reset the PVARP interval and to reset the VREF interval in the ventricular device upon detection of a premature ventricular contraction (PVC). In accordance with embodiments herein, by using triggered pulses as a distributed communication scheme between separate atrial and ventricular devices, the devices operate at virtually the same time because of the short duration required for trigger pulses to propagate between the devices.

In accordance with certain embodiments, the pulse amp 54 (when used in a ventricular device) may be designed to detect subthreshold trigger pulses that are delivered at the end of an AV delay, in order to trigger ventricular pacing. In another embodiment, atrial pacing pulses or a communications trigger pulse may be used to trigger an AV delay in the ventricular device such that the ventricular device alone, or both the atrial and ventricular devices utilize AV delay timers. When the ventricular device includes an AV delay timer, then the ventricular device may discriminate between atrial paced events and special paired communication trigger pulses that are initiated by an intrinsic atrial sensed event, thereby allowing the paced AV delay and the sensed AV delay to be set, respectively. As an example, the sensed AV delays may be shorter (e.g., 60 milliseconds shorter) than the paced AV delay.

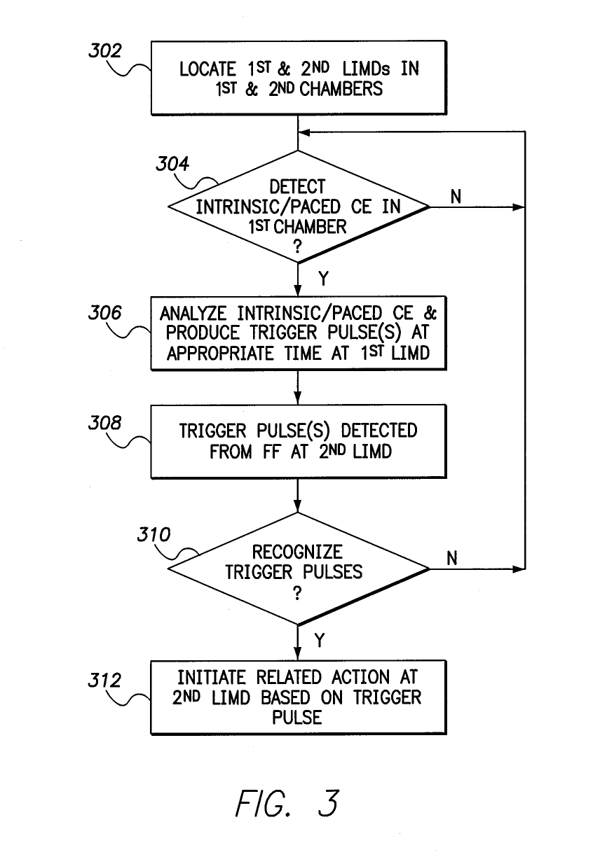

FIG. 3 illustrates a method in accordance with an embodiment herein for managing communication between LIMDs in connection with providing select modes of operation, such as DDD and WI modes. Beginning at 302, at least first and second LIMDs are configured to be implanted entirely within first and second corresponding chambers of the heart. The first and second LIMDs are positioned such that electrodes engage local tissue of interest in the corresponding first and second chambers. At 304, the first LIMD begins detecting at least one of intrinsic and paced cardiac events that originate in the corresponding first chamber. For example, an atrial device would detect intrinsic and paced atrial events, while a ventricular device would detect intrinsic and paced ventricular cardiac events.

At 306, the intrinsic and paced cardiac events are analyzed at the first LIMD and based thereon, at least one trigger pulse is produced from an electrode or electrodes of the first LIMD when an event of interest occurs in the first chamber. As noted throughout, the event of interest may represent a physiologic event occurring within the local chamber, an interval or timer that begins or ends, or some other change of state in the operation of the first LIMD (generally referred to as non-physiologic actions).

At 308, a detection operation occurs in the second chamber by the second LIMD. The second LIMD detects the trigger pulse that was produced by the first LIMD. The trigger pulse originates in the far field associated with the first chamber in which the first LIMD is located. The trigger pulse has a predetermined pattern that is configured to indicate that an event of interest has occurred. Optionally, different patterns may be defined for different trigger pulses to be uniquely associated with separate events of interest or to be uniquely associated with classes of events of interest.

At 310, the method determines whether the trigger event is recognized to indicate an occurrence of an event of interest in the remote chamber. If so, at 312, a related action is taken in the corresponding local chamber. For example, when the remote chamber represents an atrium, the operation at 312 would initiate a corresponding and related action in a ventricle.

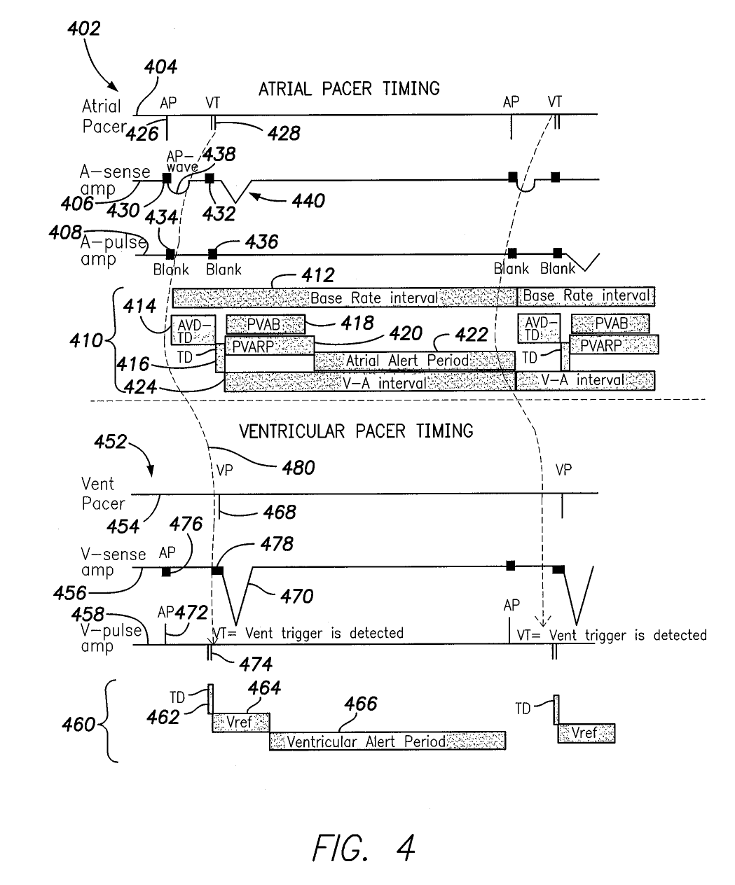

FIG. 4 illustrates a timing diagram followed in accordance with a distributed communications embodiment. The timing diagram of FIG. 4 shows the coordination between a pair of LIMDs 14, 16 configured to operate jointly in a DDD mode. Within the timing diagram, a timing diagram 402 is illustrated in connection with the atrial device, while a timing diagram 452 is illustrated in connection with the ventricular device.

In the example of FIG. 4, the atrial and ventricular devices are set in a DDD mode with preprogrammed base rates. The timing diagrams of FIG. 4 indicate the operation of the atrial and ventricular devices when the heart's intrinsic atrial rate is lower (slower) than the programmed atrial base rate. For example, if the atrial device is programmed with a base rate of 60 beats per minute (bpm), and the intrinsic atrial rate is 50 beats per minute, the behavior exhibited in timing diagram 402 would potentially occur.

The atrial timing diagram 402 illustrates an atrial pacer channel 404, an atrial cardiac sensing channel 406 and an atrial pulse sensing channel 408. Channel 404 indicates pulses generated at the electrodes of the atrial device, such as an atrial pacing pulse 426, triggering pulses 428 and the like. The cardiac sensing channel 406 indicates signals sensed at a select combination of electrodes on the atrial device when coupled to the cardiac sensing circuit (32 in FIG. 2). Within the cardiac sensing channel 406, a pair of blanking intervals 430 and 432 are denoted at which the cardiac sensing channel 32 is rendered insensitive to avoid sensing the atrial pacing pulse 426 and triggering pulse(s) 428. A region 431 denotes the atrial activity (depolarization) sensed following the atrial pacing pulse. The cardiac sensing channel 406 may detect R-waves 440 in the far field and the like.

The pulse sensing channel 408 also includes blanking intervals 434 and 436 during which the pulse sensing circuit 34 is rendered insensitive to avoid detection of the atrial pacing pulse 426 and trigger pulses 428.

The timing diagram 402 of the atrial device also indicates a series of timers 410 which may represent state machines or timers utilized by the atrial device in connection with performing DDD mode pacing. The timers 410 include a base rate interval 412, an AV time delay 414, and a time delay 416 follows the AVD-TD 414. Following the time delay 416, timers for a post-ventricular atrial blanking interval (PVAB) 418 is started, as well as a PVARP interval 420 and a ventricular atrial interval (V-A interval) 424. When the PVARP interval 420 times out, an atrial alert period 422 begins. Optionally, the timers 410 may be varied in duration, be omitted entirely or additional timing intervals may be added. In the example of FIG. 4, the timers 410 are managed within the LIMD 14 which represents the atrial pacing device. Optionally, a portion or all of the timers 410 may be implemented within or managed by the LIMD 16 representing the ventricular pacing device.

The timing diagram 452 associated with the ventricular device includes a ventricular pacing channel 454, a ventricular sensing channel 456, and a ventricular pulse sensing channel 458. The pacing channel 454 denotes stimulation pulses, such as ventricular pacing pulse 468, as well as triggering pulses (not shown in FIG. 4), which may be produced by the ventricular device.

The ventricular sensing channel 456 illustrates signals sensed at a select combination of electrodes by the ventricular device. In the example of FIG. 4, the ventricular sensing channel 456 includes blanking intervals 476 and 478 which are timed to correspond to the atrial pacing pulse 426 delivered from the atrial device and with the ventricular pacing pulse 468. The ventricular sensing channel 456 also senses the ventricular depolarization event (as denoted at 470) following delivery of the ventricular pacing pulse 468.

The pulse sensing channel 458 senses the atrial pacing pulse 472 delivered from the atrial device, as well as the trigger pulses 474 delivered by the atrial pacing device. A link 480 is denoted in dashed lines to indicate that the triggering pulses 428 delivered by the atrial device are sensed as FF trigger pulses 474 at the ventricular device. As explained herein, the delivery and sensing of trigger pulses at select times are utilized to enable communication and coordinated operation between the atrial and ventricular devices. The trigger pulses have a predetermined pattern that is configured to indicate that an event of interest has occurred in a corresponding chamber of the heart in which the atrial or ventricular device is located that generates the associated triggering pulse. Based upon the time at which a triggering pulse is delivered, as well as the device (atrial or ventricular) from which the triggering pulse is produced, such triggering pulses will have different meanings and cause the initiation of a related action from the other device.

The ventricular timing diagram 452 also indicates a collection of timers 460 which also may be implemented as state machines or timers. The timers 460 include a time delay 462 that is initiated upon detection of the triggering pulses 474. Following the time delay 462, the ventricular device activates a ventricular refractory interval 464. The ventricular refractory interval 464 represents a time period during which the ventricle is expected to remain in a refractory state. Upon conclusion of the VRef interval 464, a ventricular alert period 466 is started. During the ventricular alert period 466, an intrinsic ventricular event is not expected. When an intrinsic ventricular event occurs during a ventricular alert period 466, certain corrective actions may be taken. For example, the atrial LIMD may start the PVARP interval and the ventricular LIMD may start the Vref interval when a PVC occurs.

When comparing the atrial and ventricular timing diagrams 402 and 452, in the example of FIG. 4, it can be seen that the atrial pacing pulse 426 is detected on the ventricular pulse sensing channel 458, and is blanked out on the ventricular cardiac sensing channel 456 and atrial cardiac sensing channel 406. The atrial paced event 426 may be potentially blocked out on the V sensing channel 456 or alternatively discarded as a far field atrial pace artifact. Near the end of the AV delay 414, a ventricular triggering pulse 428 is produced across the electrodes of the atrial device. The triggering pulses 428 may be configured as a subthreshold double pulse set at an amplitude below the captured threshold associated with the atrial device. The triggering pulses 428 are detected over the pulse sensing channel 458 and used to trigger, following time delay 462, the VRef interval 464. When the triggering pulses 428 are detected over the pulse sensing channel 458 (at 474), the ventricular device triggers ventricular pacing 468 after the time delay 462. Following delivery of the ventricular pacing pulses 468, the ventricular devices enters the VRef state for the VRef interval 464. Upon determination of the VRef interval 464, the ventricular device enters an alert state during the ventricular alert period 466 in search of premature ventricular contractions.

Optionally, the first LIMD may produce an AV start trigger, as the trigger pulse, and the second LIMD may detect and recognize the AV start trigger as an instruction for the second LIMD to start an AV delay. Optionally, the second LIMD may produce a VA end trigger, as the trigger pulse, and the first LIMD may detect and recognize the VA end trigger to indicate an end of the VA delay or interval. In this last instance, the first LIMD is configured to deliver a pacing pulse upon detecting the VA end trigger, unless the first LIMD detects a corresponding intrinsic atrial event before detecting the VA end trigger.

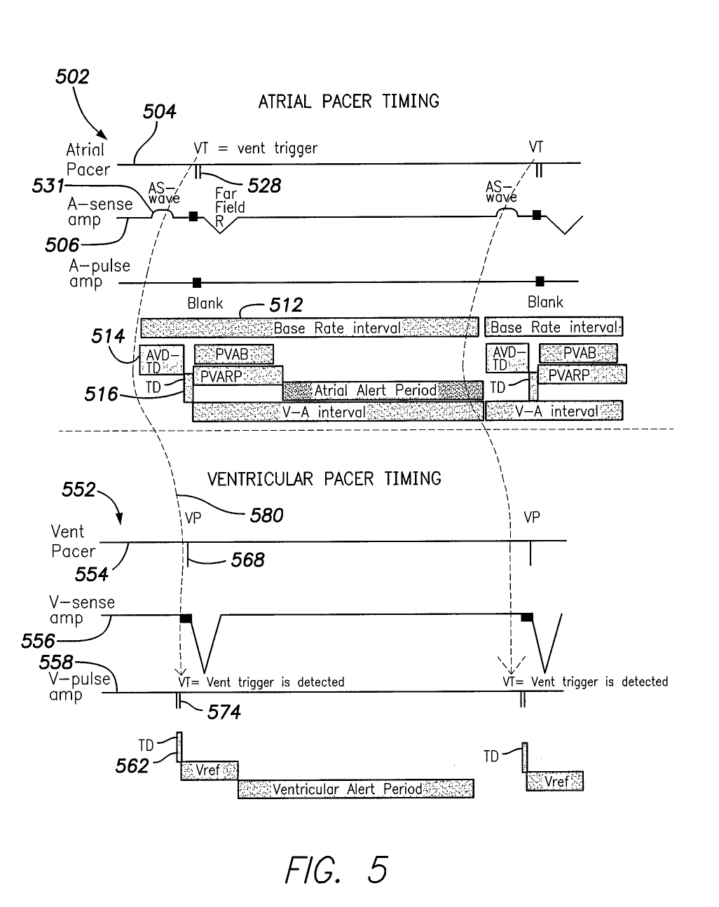

FIG. 5 illustrates timing diagrams 502, 552 that may be implemented in accordance with an embodiment. In the timing diagrams 502, 552 the atrial and ventricular devices operate in a DDD mode with a programmed base rate (e.g., 60 beats per minute) that is lower than an intrinsic atrial rate (e.g., 70 beats per minute) exhibited by a patient. Initially, the atrial sensing channel 506 detects an intrinsic P-wave 531 which causes the atrial device to initiate an AV delay 514 and a base rate interval 512. Following termination of the AV delay 514, an additional time delay 516 is started, and upon completion, the atrial device generates a triggering pulse 528 over the atrial pacing channel 504 from the electrodes.

As shown by link 580, the ventricular device detects the trigger pulses at 574 over the pulse sensing channel 558. Upon detection of the triggering pulses at 574, a time delay 562 is initiated. Upon completion of the time delay 562, a ventricular pacing pulse 568 is delivered over the pacing channel 554. It should be recognized, that if an intrinsic ventricular event were sensed over the ventricular sensing channel 556 before termination of the timing delay 562 (or before detection of the triggering pulse 574), the ventricular device would not deliver the ventricular pacing pulse 568. Following delivery of the ventricular pacing pulse 568, the remainder of the operation of the atrial and ventricular devices follows the process set forth in connection with FIG. 4 whereby various additional intervals are set to monitor for certain physiologic behavior.

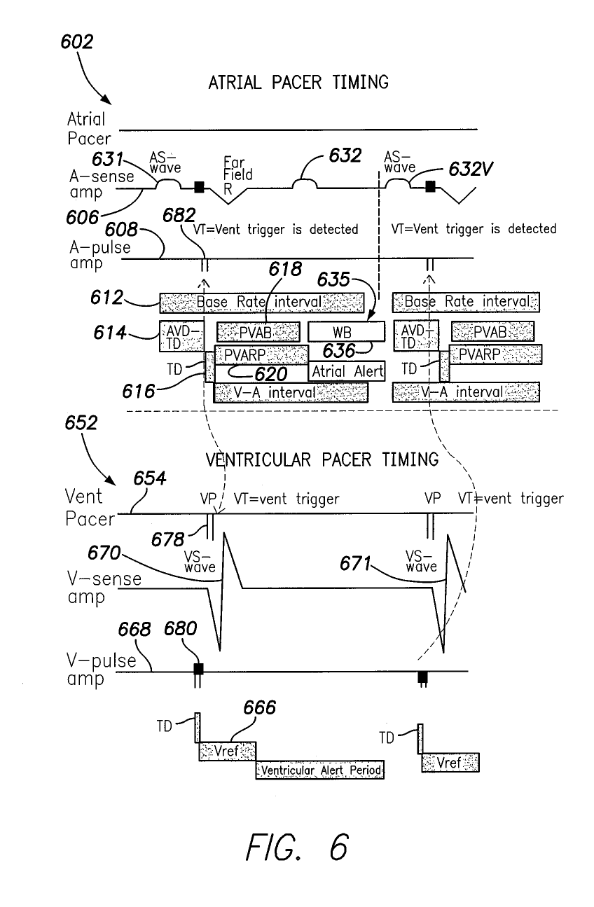

FIG. 6 illustrates timing diagrams associated with another abnormal physiologic condition. The atrial device senses an intrinsic P-wave at 631 over the atrial sensing channel 606. Upon detection of the P-wave, the atrial device initiates the base rate interval 612 and the AV delay interval 614. At the end of the AV delay interval 614, the time delay 616 is initiated. As explained in connection with the prior examples, if a ventricular event is not detected by the end of the time delay 616, the atrial device would produce a trigger pulse (similar to trigger pulse 528 in FIG. 5). However, in the example of FIG. 6, the ventricular device (as illustrated in timing diagram 652) detects an intrinsic R-wave 670 prior to completion of the timing delay 616. When the ventricular device detects the R-wave 670, the ventricular device produces a triggering pulse 678 over the ventricular pacing channel 654.

Optionally, the pulse sensing channel 668 may be inhibited by a blanking interval 680 during generation of the trigger pulse 678. The trigger pulse 678 is detected by the atrial device over the atrial pulse sensing channel 608 at the point denoted at 682. Upon detection of the trigger pulse at 682 over the atrial pulse sensing channel, the atrial device triggers reversion to the PVARP state where the PVARP interval 620 and the PVAB interval 618 are initiated. Following detection of the intrinsic R-wave 670, the ventricular device enters the VRef state at 666.

In the example of FIG. 6, the physiologic intrinsic rate is higher than the master tracking rate, where the P-waves and R-waves do not occur in a one to one relation (1<P:R<2), but a ratio of intrinsic P-waves and R-waves is less than 2:1 (P:R). The timing diagram 602 corresponds to the operation of the atrial device, while the timing diagram 652 corresponds to the operation of the ventricular device. In the example of FIG. 6, the heart experiences P-waves at a faster rate than R-waves, for example P-waves 631 and 632, occur at a faster than R-waves 670 and 671. The spacing and relations in FIG. 6 are not temporally to scale. Therefore, while the P-waves 631-632 appear to occur at an uneven interval, the P-waves 631-632 may occur evenly spaced over time, but still at a faster rate than R-waves 670-671.

The atrial device utilizes the same intervals as discussed above in connection with FIGS. 4-5, including a timer referred to as the Wenckebach (WB) interval 635. The WB interval 635 may also be tracked in connection with the other examples provided herein, but is not discussed elsewhere as the physiologic behavior in FIGS. 4-5 does not trigger any particular response relative to the WB interval 635. The WB interval 635 begins at the end of the PVARP interval 620. The WB interval 635 continues for an initial portion of the atrial alert period and ends at WB end 636.

During operation, when the atrial device, experiences the P-wave 632 which occurs during the WB interval 635, the atrial device do not “track” or respond to the P-wave in the same manner as the atrial device responded to P-wave 631. Instead, when a P-wave 632 is sensed during the WB interval 635, the atrial device “shifts” the P-wave 632 as denoted by the arrow 637 to a new position temporally aligned with the WB end 636. The atrial device shifts the P-wave 632 to create a “phantom” P-wave 632V that occurs at the end of the WB interval 635. The atrial device then operates in a standard state sequence, as explained herein, treating the phantom P-wave 632V as an “actual” P-wave.

By shifting the P-wave 632 to the end of the Wenckebach interval 635, the atrial device prevents the atrial and ventricular devices from operating in a manner that might otherwise induce device driven Wenckebach behavior. Device driven Wenckebach behavior refers to pacing the atrium and ventricle in a manner that resembles the heart block condition generally referred to as the Wenckebach phenomenon in which a pulse from the atrium periodically does not reach the ventricle and which is characterized by progressive prolongation of the P-R interval.

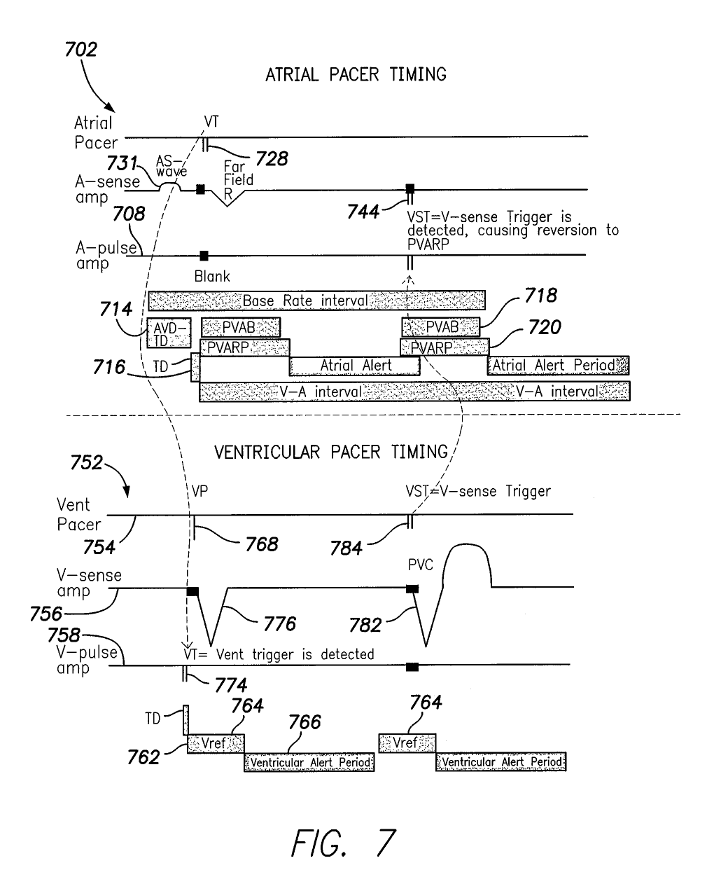

FIG. 7 illustrates timing diagrams 702 and 752 corresponding to atrial and ventricular devices operating in a DDD mode, when a premature ventricular contraction occurs. As in the example of FIGS. 5 and 6, the atrial device detects an intrinsic P-wave 731 which initiates an AV delay 714, followed by an additional time delay 716. At the end of the time delay 716, when no ventricular event is detected by the ventricular device, the atrial device generates a trigger pulse 728 which is sensed at 774 by the ventricular device over the pulse sensing channel 758. Upon the detection of the triggering signal at 774, the ventricular device produces a ventricular pacing pulse 768 over the pacing channel 754. The ventricular sensing channel 756 senses the depolarization event 776 following the pacing pulse 768. The ventricular device also initiates the VRef interval 764 following the time delay 762. Upon timeout of the VRef interval 764, a ventricular alert period interval 766 is started.

In the example of FIG. 7, the patient experiences a premature ventricular contraction (PVC) (as denoted at 782) which would normally occur during the ventricular alert period 766. Upon detection of the PVC 782, the ventricular device generates a trigger pulse 784 to inform the atrial device of the PVC. The atrial device senses the trigger pulse 784 over the full sensing channel 708 at 744. Upon detection of the PVC 782, the ventricular device reverts to and reinitiates the VRef interval 764. Upon detection of the trigger pulse at 744, the atrial device reverts to and restarts the PVARP interval 720 and the PVAB interval 718.

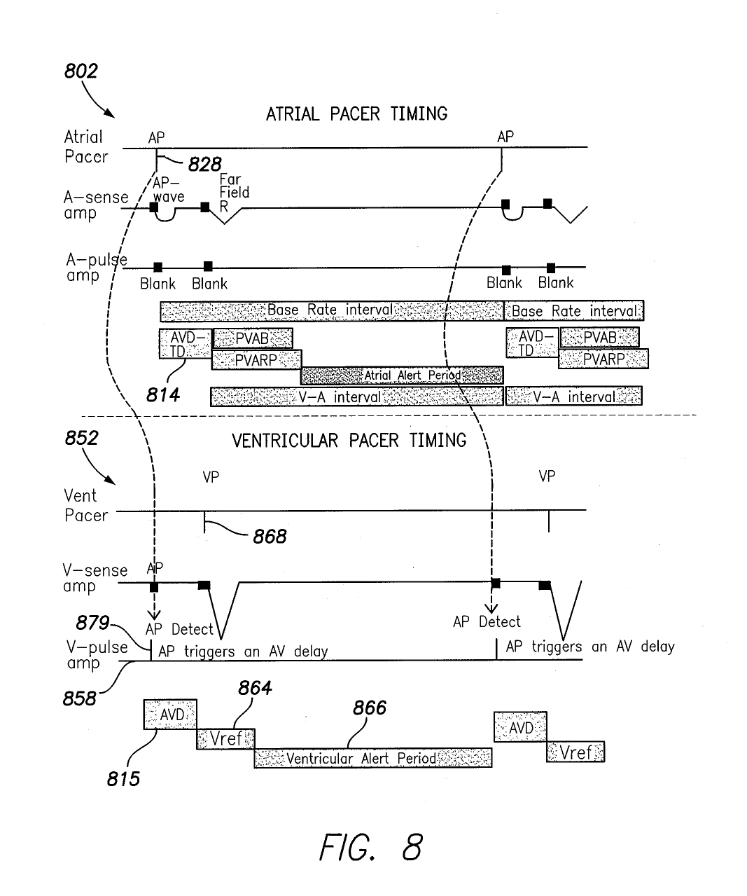

FIG. 8 illustrates timing diagrams 802 and 852 that illustrate the operation of atrial and ventricular devices configured to operate in accordance with an alternative embodiment. In the embodiment of FIG. 8, the atrial and ventricular devices are configured such that, when the atrial device generates an atrial pace event 828, the ventricular device detects the atrial pace event 828 at 879 over the pulse sensing channel 858. Upon detection of the atrial paced event (at the ventricular device), the ventricular device initiates an internal clock programmed to time an AV delay interval 815. Upon exploration of the AV delay 815 as timed in the ventricular device, the ventricular device delivers a ventricular pacing pulse 868 (unless a preceding intrinsic ventricular event is sensed during the AV delay 815). Upon generation of the ventricular paced event 868, the ventricular device enters the VRef state 864, followed by the ventricular alert period 866.

The atrial device enters the PVARP state at the end of the AV delay 814 while the ventricular device enters the VRef state after pacing. Following the VRef interval 864, the ventricular device becomes alert for PVCs by entering the ventricular alert period 866. Optionally, when the system performs P-wave tracking, a second triggering pulse may be triggered in the atrium to start a sensed AV delay that is a predetermined number of milliseconds shorter than the AV delay associated with the paced event 828. In the example of FIG. 8, the atrial paced event 828 begins AV delay timers 814 and 815 in both of the atrial and ventricular devices. The AV delays 814 and 815 expire at the same time in both the atrial and ventricular devices. In the ventricular device, the AV delay terminates with a ventricular pacing pulse being delivered, while in the atrial device, the AV delay terminates with entry into the PVARP state.

Optionally, instead of a paced atrial event, a sensed atrial event may be detected at the atrial device. When an intrinsic atrial event is sensed, the atrial device may generate a trigger pulse to signal to the ventricular device to start the AV delay associated with a sensed AV event. Optionally, the AV delay started within the ventricular device in response to detection of trigger pulses denoting an intrinsic atrial event, may differ in length from the AV delay set in the ventricular device in response to an atrial paced event. As one example, the AV delay set in the ventricular device in response to a sensed atrial event may be 60 milliseconds shorter than the AV delay set in the ventricular device in response to a paced atrial event. Optionally, other differences in length between sensed and paced AV delays may be utilized.

The PVARP interval 420 is set such that the atrial device will sense atrial events, but will not track atrial events that occur during the PVARP interval 420, in order to prevent tracking of retrograde P-waves. While atrial events that occur during the PVARP interval 420 are not tracked, such events are used to calculate the overall atrial rate. During the PVAB interval 418, the atrial sensing channel 406 is blind or blanked to prevent the atrial sensing channel 406 from sensing far field ventricular events.

During the ventricular refractory (Vref) period 464, the ventricular sensing channel 456 is disabled to prevent the ventricular device from misinterpreting a T-wave or an overly long QRS complex as a ventricular event. Otherwise, a T-wave or long QRS complex may be over-sensed and “double counted” in the ventricular device.

The LIMDs 14, 16 may operate in the presence of various types of abnormal physiologic behavior, such as Type 1 or Type 2block. Certain types of AV block manifest as a progressive prolongation of the PR interval on the electrocardiogram (ECG) on consecutive beats followed by a blocked P-wave (i.e., a ‘dropped’ QRS complex). After the dropped QRS complex, the PR interval resets and the cycle repeats. Certain types of AV block may cause the atrium to operate at a faster rate than the ventricles. For example, a heart may exhibit P-waves at a rate twice, three times or higher than the rate of the R-waves (e.g., 2:1, 3:1 and the like for P-waves-to-R-waves).

Type 2 Second-degree AV block, also known as “Mobitz II,” is characterized on a surface ECG by intermittently nonconducted P-waves not preceded by PR prolongation and not followed by PR shortening. There is usually a fixed number of non-conducted P-waves for every successfully conducted QRS complex, and this ratio is often specified in describing Mobitz II blocks. For example, Mobitz II block in which there are two P-waves for every one QRS complex may be referred to as “2:1 Mobitz II block”.

Because type I Mobitz block occurs in regular cycles, there is always a fixed ratio between the number of P-waves and the number of QRS complexes per cycle. This ratio is often specified when describing the block. For example, a Mobitz type I block which has 4 P-waves and 3 QRS complexes per cycle may be referred to as “4:3 Mobitz Type I block”.

In various embodiments, the LIMDs 14, 16 perform atrial tracking whereby the rate of the ventricular device tracks or follows the rate of the atrial device. Atrial tracking may be desirable during normal sinus acceleration for patients with heart block. In patients with heart block, the ventricular rate may not intrinsically follow the atrial rate as the atrial rate increases in connection with activity or exercise. In accordance with embodiments herein, when performing atrial tracking, the LIMDs 14, 16 communicate to increase the ventricular rate as the atrial rate increases.

However, when the atrial rate increases due to abnormal physiologic behavior (such as atrial tachycardias), in accordance with embodiments herein, the LIMDs 14, 16 will recognize that the atrial rate is excessive and will cooperate to only permit the ventricular rate to follow the atrial rate up to the maximum tracking rate (MTR) limit. In the event that the atrial rate increases beyond the MTR limit, the LIMDs 14, 16 limit the ventricular rate to the MTR limit.

The maximum tracking rate is programmable, and in general represents the fastest desirable rate that intrinsic P-waves can be tracked, or followed by paced ventricular events in a 1-to-1 ratio. As an example, the MTR limit may be programmed to 90-180 beats per minute (bpm), or as a further example to 130 bpm. When the patient experiences abnormal physiologic behavior at upper rates, the LIMDs 14, 16 may be programmed to operate in various manners. The LIMDs 14, 16 may be programmed with an upper/fastest atrial rate that will be followed by a ventricular device. When the intrinsic atrial rate exceeds an upper limit, the ventricular device may respond in various manners. For example, the ventricular device may revert to a fixed-ratio rate, in which the ventricular device paces at a rate that is less than a 1:1 ratio with the atrial rate. For example, the ventricular device may provide one ventricular paced event for every two atrial intrinsic events. Alternatively, the ventricular device may provide one ventricular paced event for every three atrial intrinsic events.

The LIMDs 14, 16 detect that the atrial rate exceeds the maximum tracking rate but remains below the sum of the AV delay and the PVARP interval, which, if left unconnected, could result in LIMDs 14, 16 inducing a device driven Wenckebach behavior. To avoid Wenckebach device driven behavior, the LIMDs 14, 16 may take a corrective action such as changing the ventricular pacing interval, initiate anti-tachycardia pacing, switch the mode of the atrial device from a DDD mode to an AAI mode, switch the mode of the ventricular device from a DDD mode to a VVI mode, and the like.

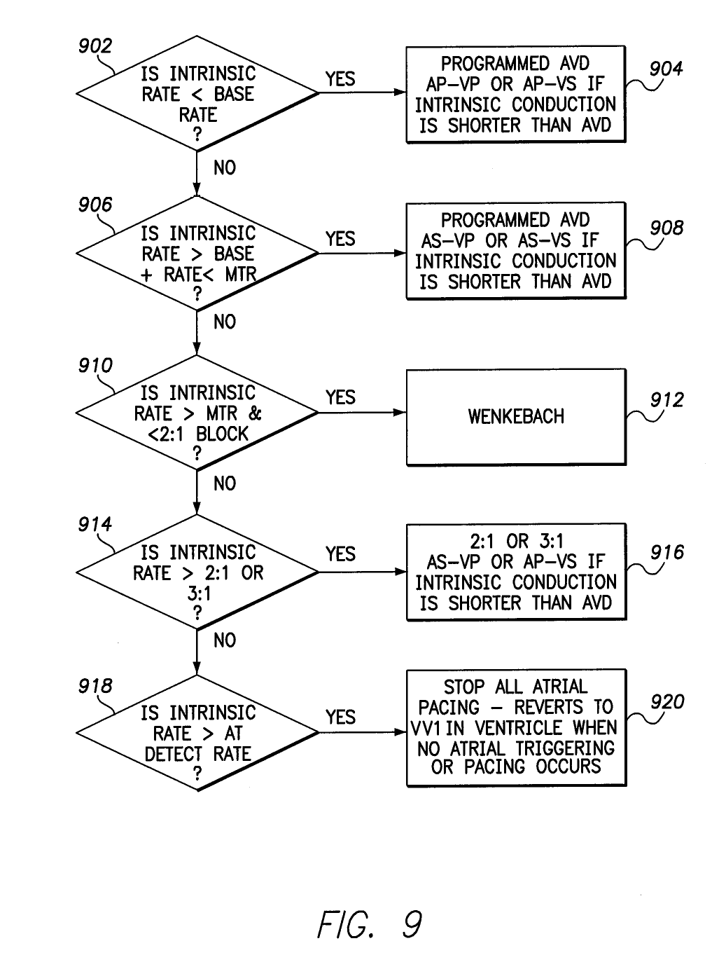

FIG. 9 illustrates a flow chart of the system behavior at different atrial rates in accordance with embodiments herein. Beginning at 902, when the intrinsic rate is below a base pacing rate programmed in the atrial device, flow moves to 904 where the atrial device performs atrial pacing (AP) according to the base pacing rate. Otherwise flow moves to 906. At 904, the atrial and ventricular devices operate in a distributed DDD mode, wherein the atrial and ventricular devices perform atrial pacing (AP) followed by ventricular pacing (VP) when the intrinsic conduction is longer than the programmed AV delay. The atrial and ventricular devices perform AP followed by ventricular sensing (VS) when the intrinsic conduction is shorter than the AV delay. At 904, the atrial and ventricular devices coordinate in the manner shown in FIG. 4. As shown in FIG. 4, the atrial device paces at 426 waits the AV delay 414 and sends a trigger pulse 428 to the ventricular device. The ventricular device paces (at 468) upon detecting the trigger pulse (at 474) unless a sensed intrinsic ventricular event has already occurred at the appropriate time.

At 906, when the intrinsic rate is greater than the base rate but less than the maximum tracking rate, then flow moves to 908. Otherwise, flow moves to 910. At 908, the atrial and ventricular devices operate, as in FIGS. 5 and 6, with the atrial device inhibited as intrinsic atrial sensed (AS) events are detected at sufficiently fast rate to suppress atrial pacing. The AS events are followed by ventricular pacing (VP) when intrinsic conduction is longer than the programmed AV delay. The atrial and ventricular devices perform AS followed by ventricular sensing (VS) when the intrinsic conduction is shorter than the AV delay.

At 910, when the intrinsic atrial rate is greater than the MTR and the ratio of atrial sensed events to ventricular sensed events is less than 2:1 (2:1 block), then flow moves to 912. Otherwise flow moves to 914. At 912, the atrial and ventricular devices cooperate to avoid inducing a Wenckebach device driven behavior. As explained in connection with FIG. 6, atrial rate exceeds the MTR and the atrial device experiences a P-wave 632 (FIG. 6) that occurs during the WB interval 635, the atrial device do not track the P-wave. Instead, the atrial device shifts the P-wave 632 to form a phantom P-wave 632V that temporally aligns with the end of the WB interval 635. The atrial device treats the phantom P-wave 632V as a normal intrinsic P-wave and resets all timers based thereon. By shifting the P-wave 632 to the end of the Wenckebach interval 635, the system avoids device driven Wenckebach behavior.

At 914, when the intrinsic atrial rate is sufficiently high such that two or three atrial events occur for each single ventricular event (2:1 block or 3:1 block), then flow moves to 916. Otherwise flow moves to 918. At 916, the atrial and ventricular devices operate such that, when the atrial device senses an AS, the ventricular device generates a VP. Alternatively, when the atrial device delivers an AP, the ventricular device senses a VS. In the situation experienced at 916, a portion of the P-waves occur during the PVARP interval and thus are ignored. For example, when the heart exhibits 2:1 block, then ½ of the P-waves may be ignored (if occurring during the PVARP interval). Similarly, when the heart exhibits 3:1 block, then ⅔ of the P-waves may be ignored.

At 918, when the intrinsic atrial rate is greater than the atrial tachycardia threshold, then flow moves to 920. At 920, the atrial device switches from the DDD mode and terminates/ceases pacing and the ventricular device switches from the DDD mode to a WI mode to operate as a stand alone device no longer responding to trigger pulses from the atrial device.

The atrial and ventricular devices repeatedly the operations of 902-920 iteratively such that when the state of the heart's behavior changes, the atrial and ventricular devices are able to switch modes and/or switch between coordination for different behavior. For example, when the atrial device shuts off and the ventricular device operates in WI mode, it may do so for a select amount of time, a select number of cardiac cycles or until a select change in the heart rate is detected. Thereafter, the atrial device may reactivate and the atrial and ventricular devices return to cooperative operation in the DDD mode.

Similarly, when the atrial and ventricular devices are cooperating to operate at 904, 908, 912, or 916, they may do so for one cardiac cycle, a select amount of time, a select number of cardiac cycles or until a select change in the heart rate is detected. As one example, the process of FIG. 9 may be repeated every cardiac cycle, or periodically after a select number of cardiac cycles.

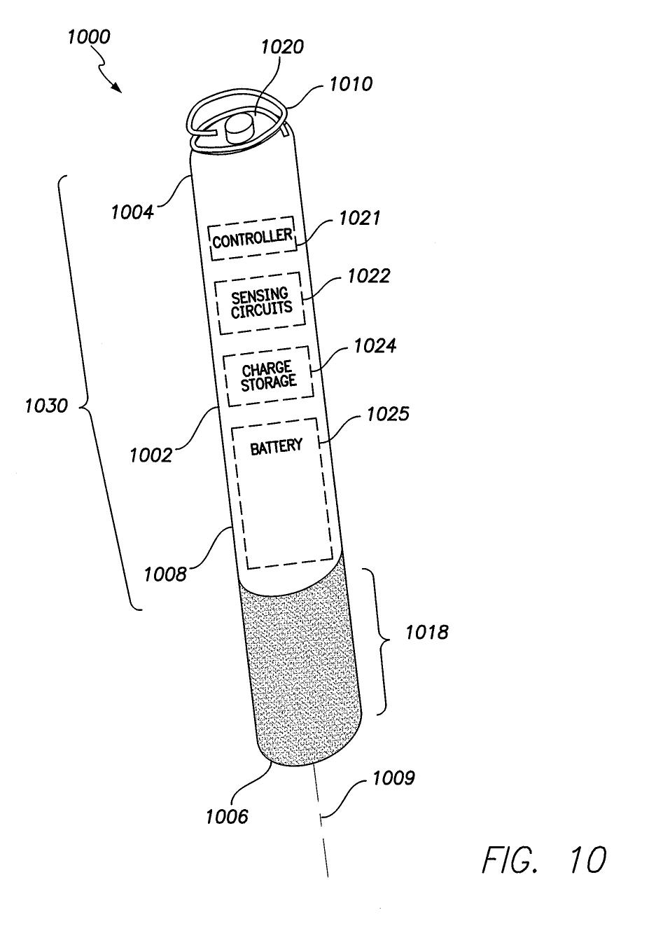

FIGS. 10 illustrate the LIMD 1000 in more detail. The LIMD 1000 comprises a housing 1002 having a distal base 1004, a distal bottom end 1006, and an intermediate shell 1008 extending between the distal base 1004 and the distal top end 1006. The shell 1008 is elongated and tubular in shape and extends along a longitudinal axis 1009. The LIMD 1000 includes a battery 1025 for power supply.

The base 1004 includes one or more electrodes 1020 securely affixed thereto and projected outward. For example, the outer element 1010 may be formed as large semi-circular spikes or large gauge wires that wrap only partially about the inner electrode 1020. The element 1010 is wound around electrode 1020. The element 1010 may be used for exclusively for fixation of the pacer to the tissue. In this case it would be inactive electrically and may be coated with an insulator like parylene or may be simply not connected to the case or any associated circuitry. Alternatively, the element 1010 may also be used as an electrode to pick up the local potentials from the tissue surrounding the electrodes 1020 and 1010. This allows for exclusive detection of electrograms from the local tissue (in the same chamber as the LIMD 1000) such that only ventricular activity is detected by the ventricle LIMD atrial activity can be exclusively detected by the atrium LIMD. This selective sensing is useful for excluding ventricular depolarization in the atrium. Avoiding sensing of far field R-waves at an atrial LIMD enables atrial event tracking and sensing of atrial arrhythmias with little or no confounding of atrial sensing due to far field R-waves. When the atrial LIMD is “desensitized” to ventricular far field signals, the timing of the atrial LIMD may be updated to set the PVAB to be very short.

Conversely sensing between 1010 and 1020 in the ventricle excludes any sensing of atrial activity in the ventricle even if the ventricular LIMD is located in tissue proximate to the atrium, like the right ventricular septum or vestibule of the RV. This prevents inappropriate detection by the ventricular LIMD of atrial depolarizations as ventricular events, which might otherwise detract from the processes described herein.

Included at the proximal end of the LIMD is an electrode 1018 that includes the distal end 1006. Electrode 1018 is electrically connected to the sensing circuits 1022 and is used to perform pulse sensing. The pulse sensing is performed between the stimulation cathode 1020 and the electrode 1018. Because the cathode 1020 and electrode 1018 are relatively widely separated, it makes it possible for the LIMD to communicate using the trigger pulses and pacing pulses at relatively large distances. This is true for communication from the ventricle to the atrium as well as communication from the atrium to the ventricle. In between the cathode 1020 and the proximal electrode 1018 is an insulated region 1030 that separates the pulse sensing electrodes 1020 and 1018. The region 1030 may be insulated with a parylene coating.

Pulses are generated by the Charge Storage circuit 1024 and are emitted between the cathode 1020 and the anode 1018 for both stimulation and communication between the devices. Because of the relatively large separation between these electrodes 1018 and 1020 the dipole field generated in the tissue is large as well and this also facilitates communication using pacing pulses and triggered pulses between the devices. So the relatively large separation between the electrodes 1020 and 1018 facilitates both reception and transmission of the information carried on the pulses over relative large distances in the body. For example, the distance between electrodes 1020 and 1018 may be one-half to two-thirds of the overall length of the LIMD 1000 (e.g., over 10 mm, 5-20 mm, up to 30 mm).

If the element 1010 is electrically active, it may also be used for sensing pulses using the sensing circuits 1022 and in addition it may be used for providing triggered pulses. In addition if pacing is performed between the 1010 and 1020, then communication between the chambers using the pacing pulses is avoided because the local dipole is so small that there is little or no signaling to the remote LIMD doing sensing. This is useful if the atrial LIMD performs atrial tachycardia pacing.

The LIMD 1000 includes a charge storage unit 1024 and sensing circuit 1022 (including cardiac event sensing circuitry 32 and pulse sensing circuitry 34) within the housing 1002. The sensing circuit 1022 senses intrinsic activity, while the charge storage unit 1024 stores high or low energy amounts to be delivered in one or more stimulus pulses. The sensing circuit 1022 senses intrinsic and paced events, as well as trigger pulses. The electrodes 1010, 1020 may be used to deliver lower energy or high energy stimulus, such as pacing pulses, cardioverter pulse trains, and the like. The electrodes 1010, 1018 also sense trigger pulses as described herein. The electrodes 1010, 1018 may also be used to sense electrical activity, such as physiologic and pathologic behavior and events and provide sensed signals to the sensing circuit 1022. The electrodes 1010, 1018 are configured to be joined to an energy source, such as a charge storage unit 1024. The electrodes 1010, 1018 receive stimulus pulse(s) from the charge storage unit 1024. The electrodes 1010, 1018 are configured to deliver high or low energy stimulus pulses to the myocardium.

The LIMD 1000 includes a controller 1021, within the housing 1002 to cause the charge storage unit 1024 to deliver activation pulses through each of the electrodes 1010, 1018 in a synchronous manner, based on information from the sensing circuit 1022, such that activation pulses delivered from the inner electrode 1010 are timed to initiate activation in the adjacent chamber. The controller 1021 performs the various operations described herein in connection alternative embodiments for the systems and the methods. The stimulus pulses are delivered synchronously to local and distal activation sites in the local and distal conduction networks such that stimulus pulses delivered at the distal activation site are timed to cause contraction of the adjacent chamber in a predetermined relation to contraction of the local chamber.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the inventive subject matter without departing from its scope. While the dimensions and types of materials described herein are intended to define the parameters of the inventive subject matter, they are by no means limiting and are exemplary embodiments. Many other embodiments will be apparent to one of ordinary skill in the art upon reviewing the above description. The scope of the inventive subject matter should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112(f) unless and until such claim limitations expressly use the phrase “means for” followed by a statement of function void of further structure.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the inventive subject matter without departing from its scope. While the dimensions and types of materials described herein are intended to define the parameters of the inventive subject matter, they are by no means limiting and are exemplary embodiments. Many other embodiments will be apparent to one of ordinary skill in the art upon reviewing the above description. The scope of the inventive subject matter should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112(f) unless and until such claim limitations expressly use the phrase “means for” followed by a statement of function void of further structure.

What is claimed is:

1. A distributed leadless implantable system, comprising:
first and second leadless implantable medical devices (LIMD) configured to be configured to be implanted entirely within first and second chambers of the heart, each of the first and second LIMDs comprising:
a housing having a proximal end configured to engage local tissue of interest in a local chamber;
electrodes located along the housing;
cardiac sensing circuitry configured to detect intrinsic and paced cardiac events occurring in a near field associated with the local chamber;
a controller configured to analyze the intrinsic and paced events and, based thereon, produce a trigger pulse at the electrodes when an event of interest occurs in the local chamber;
pulse sensing circuitry configured to detect at least one of a paced event or a trigger pulse occurring in a far field (FF), where the paced event or trigger pulse originates in a remote chamber that differs from the corresponding local chamber, the trigger pulse having a predetermined pattern configured to indicate that an event of interest has occurred in the remote chamber; and
the controller configured to recognize the at least one of the paced event or trigger pulse to indicate an occurrence of the event of interest in the remote chamber and, in response thereto, initiate a related action in the local chamber.

2. The system of claim 1, wherein the first LIMD is located in an atrium and the second LIMD is located in a ventricle, the first LIMD producing an AS/AP trigger, as the trigger pulse, to indicate that an atrial sensed (AS) event or atrial paced (AP) event occurred.

3. The system of claim 1, wherein the first LIMD is located in an atrium and the second LIMD is located in a ventricle, the second LIMD producing a VS/VP trigger, as the trigger pulse, to indicate that a ventricular sensed (VS) event or ventricular paced (VP) event occurred.

4. The system of claim 1, wherein the first LIMD is located in an atrium and the second LIMD is located in a ventricle, the first LIMD producing an AV end trigger, as the trigger pulse, the second LIMD detecting and recognizing the AV end trigger to indicate an end of an AV delay, the second LIMD configured to deliver a pacing pulse upon detecting the AV end trigger, unless the second LIMD detected a corresponding intrinsic ventricular event before detecting the AV end trigger.

5. The system of claim 1, wherein the first LIMD is located in an atrium and the second LIMD is located in a ventricle, the first LIMD producing an AV start trigger, as the trigger pulse, the second LIMD detecting and recognizing the AV start trigger as an instruction for the second LIMD to start an AV delay.

6. The system of claim 1, wherein the first LIMD is located in an atrium and the second LIMD is located in a ventricle, the second LIMD producing a VA end trigger, as the trigger pulse, the first LIMD detecting and recognizing the VA end trigger to indicate an end of a VA delay, the first LIMD configured to deliver a pacing pulse upon detecting the VA end trigger, unless the first LIMD detected a corresponding intrinsic atrial event before detecting the VA end trigger.

7. The system of claim 1, wherein the first LIMD is located in an atrium and the second LIMD is located in a ventricle, the second LIMD producing a post ventricular-atrial refractory period (PVARP) start trigger, as the trigger pulse, the first LIMD detecting and recognizing the PVARP start trigger as an instruction for the first LIMD to start a PVARP delay.

8. The system of claim 1, wherein the first LIMD is located in an atrium and the second LIMD is located in a ventricle, the first LIMD being set in an AAI mode with a first base rate and the second LIMD being set in a WI mode with a second base rate that is lower than the first base rate, the second LIMD ignoring atrial pulses from the first LIMD.

9. The system of claim 1, wherein the controller produces the trigger pulses formatted in a manner that distinguishes the trigger pulses from pacing pulses based on at least one of amplitude, pulse width, inter-pulse delay, pulse frequency, or pulse morphology.

10. The system of claim 1, wherein the first LIMD is located in an atrium and the second LIMD is located in a ventricle and wherein, when the first LIMD in the atrium is turned off or fails, the second LIMD in the ventricle functions in a VVI pacing mode.

11. A method for providing an implantable cardiac system, the method comprising:
providing first and second leadless implantable medical devices (LIMD) configured to be implanted entirely within first and second chambers of the heart, where the first and second LIMD have electrodes on housings thereof to engage local tissue of interest in a corresponding first and second chambers;
detecting, at the first LIMD, at least one of intrinsic or paced events originating in the first chamber;
analyzing, at the first LIMD, the intrinsic and paced events and, based thereon, produce a trigger pulse at the electrodes of the first LIMD when an event of interest occurs in the first chamber;
detecting, at the second LIMD in the second chamber, at least one of a paced event or the trigger pulse that was produced by the first LIMD in a far field (FF) within the first chamber, where the at least one of the paced event or trigger pulse originates in a remote chamber that differs from the corresponding local chamber, the trigger pulse having a predetermined pattern configured to indicate that an event of interest has occurred in the remote chamber; and
the controller configured to recognize the at least one of the paced event or trigger pulse to indicate an occurrence of the event of interest in the remote chamber and, in response thereto, initiate a related action in the local chamber.

12. The method of claim 11, further comprising locating the first LIMD in an atrium and locating the second LIMD in a ventricle, producing an AS/AP trigger from the first LIMD, as the trigger pulse, to indicate that an atrial sensed (AS) event or atrial paced (AP) event occurred.

13. The method of claim 11, further comprising locating the first LIMD in an atrium and locating the second LIMD in a ventricle, producing a VS/VP trigger from the second LIMD, to indicate that a ventricular sensed (VS) event or ventricular paced (VP) event occurred.

14. The method of claim 11, further comprising producing an AV end trigger, detecting and recognizing the AV end trigger to indicate an end of an AV delay, delivering a pacing pulse upon detecting the AV end trigger, unless a corresponding intrinsic ventricular event is detected before detecting the AV end trigger.

15. The method of claim 11, further comprising producing an AV start trigger, as the trigger pulse, and detecting and recognizing the AV start trigger as an instruction for the second LIMD to start an AV delay.

16. The method of claim 11, further comprising producing a VA end trigger, as the trigger pulse, detecting and recognizing the VA end trigger to indicate an end of a VA delay, delivering a pacing pulse upon detecting the VA end trigger, unless the first LIMD detected a corresponding intrinsic atrial event before detecting the VA end trigger.

17. The method of claim 11, further comprising producing a post ventricular-atrial refractory period (PVARP) start trigger, as the trigger pulse, and detecting and recognizing the PVARP start trigger as an instruction for the first LIMD to start a PVARP delay.

18. The method of claim 11, further comprising setting the first LIMD in an AAI mode with a first base rate and setting the second LIMD in a VVI mode with a second base rate that is lower than the first base rate, the second LIMD ignoring atrial pulses from the first LIMD.

19. The method of claim 11, further comprising, when the first LIMD is turned off or fails, changing a mode of operation of the second LIMD from a DDD pacing mode to a VVI pacing mode.
